(19) United States
(12) Patent Application Publication
(10) Pub. No.: US 2024/0076710 A1
(43) Pub. Date: Mar. 7, 2024

(54) NANOWIRED FORMATE DEHYDROGENASE SYSTEM FOR REDUCTION OF CARBON DIOXIDE TO FORMATE

(71) Applicant: Alliance for Sustainable Energy, LLC, Golden, CO (US)

(72) Inventors: Roman BRUNECKY, Arvada, CO (US); Chaiwat ENGTRAKUL, Louisville, CO (US); Carolyn Elizabeth LUBNER, Lakewood, CO (US)

(21) Appl. No.: 18/461,351

(22) Filed: Sep. 5, 2023

Related U.S. Application Data
(60) Provisional application No. 63/374,464, filed on Sep. 2, 2022.

(51) Int. Cl. C12Q 1/00; C25B 3/07; C25B 3/26; C25B 11/054; C25B 11/061; C25B 11/095

(52) U.S. Cl. CPC C12Q 1/005; C25B 3/07; C25B 3/26; C25B 11/054; C25B 11/061; C25B 11/095

(57) ABSTRACT

Disclosed herein are systems, methods and compositions of matter for engineered formate dehydrogenases electrocatalytically driven in reverse to fix CO₂ to formate, an electrode interface composed of self-assembled monolayers (SAM), and a nanowire link between the electrode and the enzyme's iron sulfur (FeS) cluster (engineered) to specifically link the iron sulfur cluster to the electrode. Engineering the interface between the enzyme and the electrode enables a high degree of activity and function compared to just an enzyme immobilized on the electrode. This interface enables specific orientational control of the enzyme and allows for separation of the enzyme from the electrode surface to allow retention of its native hydrodynamic radius. The transfer of electrons via the nanowire to the FeS cluster allows the enzyme to utilize its native electron transfer pathways. Moreover, wiring the FeS cluster directly to the electrode avoids the kinetic bottleneck of a soluble mediator and thus allows the wired enzyme to function more efficiently.

### CROSS-REFERENCE TO RELATED APPLICATIONS

This application claims priority under 35 U.S.C. § 119 to provisional patent application No. 63/374,464 filed on 2 Sep. 2022, the contents of which are hereby incorporated in their entirety.

### CONTRACTUAL ORIGIN

The United States Government has rights in this invention under Contract No. DE-AC36-08GO28308 between the United States Department of Energy and the Alliance for Sustainable Energy, LLC, the Manager and Operator of the National Renewable Energy Laboratory.

### BACKGROUND

The current state of the art in enzyme immobilization is to immobilize enzymes directly onto electrodes, which leads to problems such as orientational control, where most of the enzyme may be inactivated by the interaction with the electrode (non-native dynamics) or may be in an unfavorable orientation for catalysis. Another approach is to wrap the enzyme in various electroactive polymers, that again do not provide direct electron transfer and have no orientational control over the enzyme and may reduce native enzyme breathing motions which diminishes enzyme activity.

### SUMMARY

In an aspect, disclosed herein is a method for the reduction of carbon dioxide to formate comprising linking a FeS cluster within an engineered formate dehydrogenase enzyme to a thiolate linker on a first end and linking the thiolate linker on a second end to an electrode wherein the linked enzyme is exposed to carbon dioxide. In an embodiment, The method of claim 1 wherein formate is produced at a rate of 0.16 mg of formate per hour. In an embodiment, the formate is produced at a rate of 0.16 mg of formate per hour for 5 hours. In an embodiment, the electrode comprises gold. In an embodiment, the engineered formate dehydrogenase enzyme comprises a mutation from cysteine to glycine. In an embodiment, the mutation from cysteine to glycine is in a cysteine that binds to the FeS cluster in the corresponding native formate dehydrogenase enzyme.

In an aspect, disclosed herein is a system for the reduction of carbon dioxide to formate comprising a FeS cluster within an engineered formate dehydrogenase enzyme linked to a thiolate linker on a first end and wherein the thiolate linker is linked on a second end to an electrode.

In another aspect, disclosed herein is a composition of matter comprising a FeS cluster within an engineered formate dehydrogenase enzyme linked to a thiolate linker on a first end and wherein the thiolate linker is linked on a second end to an electrode.

Other objects, advantages, and novel features of the present invention will become apparent from the following detailed description of the invention when considered in conjunction with the accompanying drawings.

### DESCRIPTION OF THE DRAWINGS

FIG. 1 depicts an overall scheme of an embodiment of the system and compositions disclosed herein.

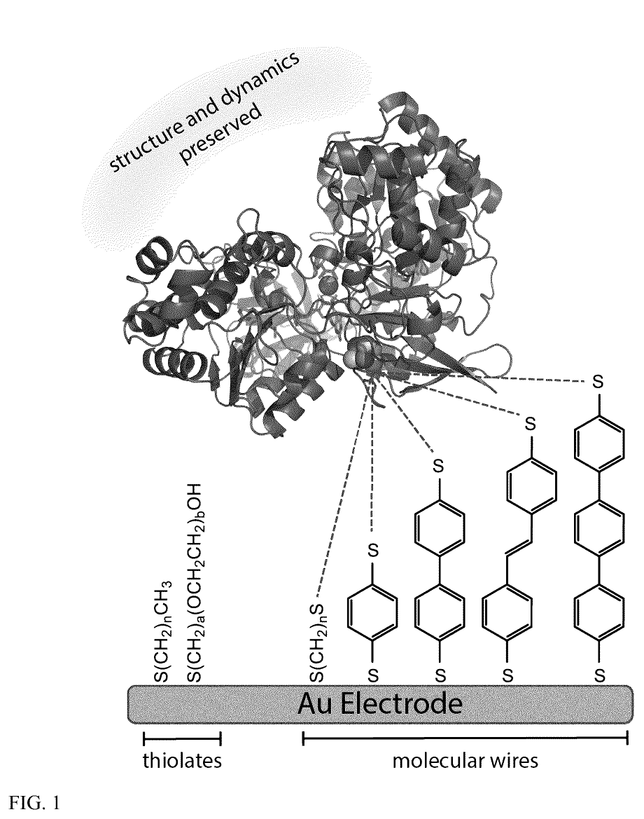

FIG. 2 depicts an embodiment of a FeS ligand swap strategy disclosed herein.

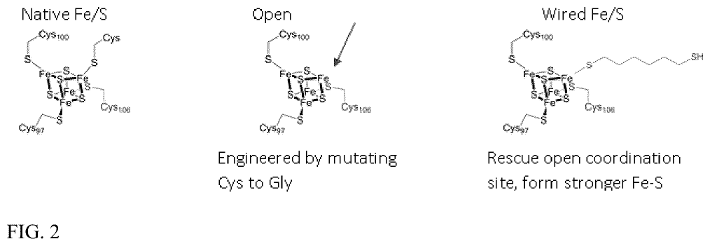

FIG. 3 depicts cyclic voltammetry traces of formate dehydrogenase (FDH) immobilized on the electrode.

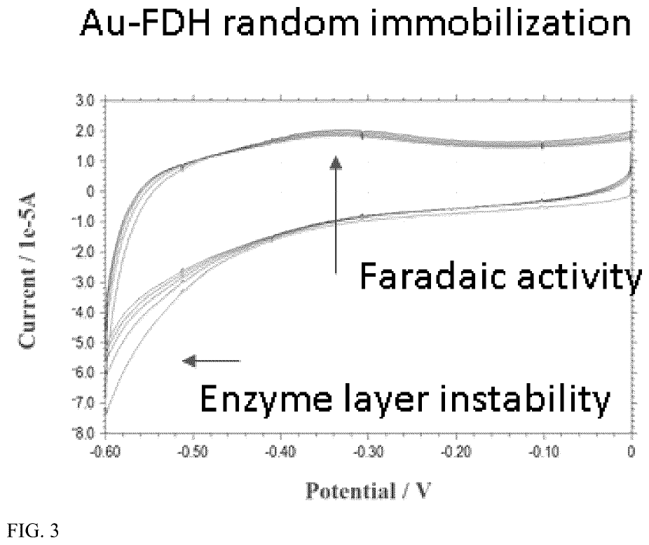

FIGS. 4a and 4b depict cyclic voltammetry traces of a SAM/nanowire arrangement illustrating (as depicted in FIG. 4a) much larger current density compared to the immobilized case, demonstrates the electron transfer process is reversible and approximately same magnitude peaks, i.e. same electron transfer rate forward and reverse. As depicted in FIG. 4b, stability of the layer after a 5-hour runtime shows some internal rearrangements of the monolayer. Without being limited by theory, this may be due to potential driven reorganization of the FDH/SAM, thus improving the molecular environment of the FDH redox center and bridge structure (alkane thiol) to the Au electrode.

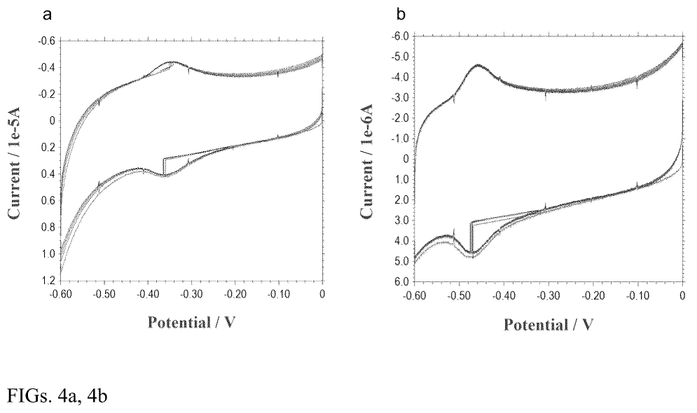

FIG. 5 depicts formate production over time using an embodiment of systems disclosed herein.

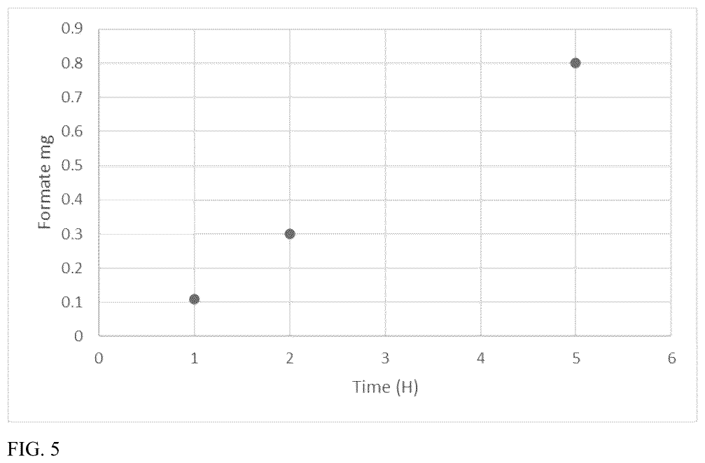

### DETAILED DESCRIPTION

In cells, soluble formate dehydrogenase (FDH) enzymes normally function in biological systems to take formate to CO₂, in doing so they generate NADH. In an embodiment, this invention is composed of several parts: an engineered FDH electrocatalytically driven in reverse to fix CO₂ to formate, an electrode interface composed of self-assembled monolayers (SAM), and a nanowire link between the electrode and the enzyme's iron sulfur (FeS) cluster (engineered) to specifically link the iron sulfur cluster to the electrode. Driving the enzyme in reverse, as well as the design for engineering the FeS cluster to be wired have been previously reported in the literature. Hence, in an embodiment, the invention deals with engineering the interface between the enzyme and the electrode which enables a high degree of activity and function compared to just an enzyme immobilized on the electrode. This interface enables specific orientational control of the enzyme and allows for separation of the enzyme from the electrode surface to allow retention of its native hydrodynamic radius. The transfer of electrons via the nanowire to the FeS cluster allows the enzyme to utilize its native electron transfer pathways. Moreover, by wiring the FeS cluster directly to the electrode we eliminate a kinetic bottleneck of a soluble mediator allowing the enzyme to function more efficiently.

Current technologies to fix CO₂ are basically trees and terrestrial plants as well as cyanobacteria; the main complex enzyme system used to fix CO₂ is Rubisco, which has a fix rate of 3-5 CO₂/sec per enzyme. FDH rates reported in the literature driven in reverse on electrodes are on the order of 100-500 CO₂/sec per enzyme. The forward rates of these enzymes are on the order of 1500+ formate to CO₂/sec. We have shown that in our experimental system we can drive both the forward and reverse electron transfer to the enzyme at the same rate with the same peak areas, implying the overall electron transfer rates in our system are equal and bi-directional. In addition, this method of immobilizing and stabilizing enzymes will likely work on other redox proteins that use an FeS cluster, i.e. nitrogenases, to enable room temperature Haber-Bosch processes, or immobilizing many other types of redox enzymes for use in cell-free systems and pathways.

Fixation of CO₂ by traditional enzyme complexes such as Rubisco, is both complex and slow and relies on soluble redox mediators. By using an FDH enzyme driven in reverse we can achieve orders of magnitude greater rates of CO₂ fixation to product vs Rubisco.

Keeping the enzyme in a favorable soluble environment through the use of both nanowires to tether the enzyme to the electrode at a favorable standoff distance, and SAM's to provide an “insulator” between the enzyme and electrode to prevent the enzyme from adsorbing to and denaturing on the surface of the electrode.

The nanowire is specifically wired to the FeS cluster located within the enzyme. This ensures all enzymes attached to the electrode are electrochemically active. The combination of SAMs and nanowires allows us to optimize the enzyme orientation near a surface to maximize both native enzyme breathing motions and electrodynamics.

Additionally, utilization of a nanowire to transfer electrons to the FeS cluster removes one important kinetic limitation found in natural systems, ie the requirement of binding both the substrate CO₂ in the active site, and also the soluble redox mediator (NAD(H)). In our case we are only limited by the CO₂ binding in the active site because the use of the nanowire removes the constraints of NAD(H) binding and coordination.

The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

What is claimed is:

1. A method for the reduction of carbon dioxide to formate comprising linking a FeS cluster within an engineered formate dehydrogenase enzyme to a thiolate linker on a first end and linking the thiolate linker on a second end to an electrode wherein the linked enzyme is exposed to carbon dioxide.

2. The method of claim 1 wherein formate is produced at a rate of 0.16 mg of formate per hour.

3. The method of claim 1 wherein formate is produced at a rate of 0.16 mg of formate per hour for 5 hours.

4. The method of claim 1 wherein the electrode comprises gold.

5. The method of claim 1 wherein the engineered formate dehydrogenase enzyme comprises a mutation from cysteine to glycine.

6. The method of claim 5 wherein the mutation from cysteine to glycine is in a cysteine that binds to the FeS cluster in the corresponding native formate dehydrogenase enzyme.

7. A system for the reduction of carbon dioxide to formate comprising a FeS cluster within an engineered formate dehydrogenase enzyme linked to a thiolate linker on a first end and wherein the thiolate linker is linked on a second end to an electrode.

8. The system of claim 7 wherein formate is produced at a rate of 0.16 mg of formate per hour.

9. The system of claim 7 wherein formate is produced at a rate of 0.16 mg of formate per hour for 5 hours.

10. The system of claim 7 wherein the electrode comprises gold.

11. The system of claim 7 wherein the engineered formate dehydrogenase enzyme comprises a mutation from cysteine to glycine.

12. The system of claim 11 wherein the mutation from cysteine to glycine is in a cysteine that binds to the FeS cluster in the corresponding native formate dehydrogenase enzyme.

13. A composition of matter comprising a FeS cluster within an engineered formate dehydrogenase enzyme linked to a thiolate linker on a first end and wherein the thiolate linker is linked on a second end to an electrode.

14. The composition of matter of claim 13 wherein the FeS cluster is linked to a thiolate linker on a first end through a glycine residue resulting from mutating a cysteine that binds to the FeS cluster in the corresponding native formate dehydrogenase enzyme.

15. The composition of matter of claim 13 wherein the electrode comprises gold.
